(19) United States
(12) Patent Application Publication
(10) Pub. No.: US 2026/0171249 A1
(43) Pub. Date: Jun. 18, 2026

(54) METHODS AND SYSTEMS FOR OPTIMIZING DIAGNOSTIC PLATFORM WORKFLOW USING ARTIFICIAL INTELLIGENCE MODELS

(71) Applicant: KONINKLIJKE PHILIPS N.V., Eindhoven (NL)

(72) Inventors: Man Ching Cheung, Cambridge, MA (US); Jon Kamerman, Andover, MA (US)

(21) Appl. No.: 19/413,150

(22) Filed: Dec. 9, 2025

Related U.S. Application Data
(60) Provisional application No. 63/734,261, filed on Dec. 16, 2024.

(51) Int. Cl. G16H 50/20

(52) U.S. Cl. CPC G16H 50/20

(57) ABSTRACT

A diagnostic platform method (100), comprising: receiving (120), from one or more data sources, information about a subject's set of results currently being reviewed by a user via a review window of the diagnostic platform; extracting (130) data from the received information to generate contextualized data regarding the set of results; receiving (140), via a user interface of the review window of the diagnostic platform, examination data about the set of results from the user; analyzing (150), by a trained model (264), the generated contextualized data and the received examination data to generate diagnostic output about the set of results; and presenting (160), via the review window of the diagnostic platform, the generated diagnostic output.

### CROSS-REFERENCE TO RELATED APPLICATIONS

This patent application claims the priority benefit under 35 U.S.C. § 119 (e) of U.S. Provisional Application No. 63/734,261, filed on Dec. 16, 2024, the contents of which are herein incorporated by reference.

### FIELD OF THE DISCLOSURE

The present disclosure is directed generally to methods and systems for optimizing the user experience with artificial intelligence-integrated diagnostic platforms.

### BACKGROUND

Artificial intelligence (AI) models such as large-language models (LLMs) are a versatile class of deep learning neural networks, capable of retrieving and recombining data from immensely large repositories and organizing results into human-comprehensible answers or output. The most popular method to invoke an AI model such as an LLM is to provide a user-friendly chat page that simulates instant messaging applications (SMS, emails, etc.). However, when applied to workflow-oriented applications, separately invoking an AI model is deleterious to the performance of tasks. Switching to another platform separate from the workflow-oriented application, in order to engage the AI model, is inefficient.

For example, clinicians reviewing results such as lab tests, diagnostics, or other results in a diagnostic platform waste significant time switching to an AI model, interacting with that AI model, and then switching back to the diagnostic platform. It may also be challenging to import the results of the interaction from the AI model back into the diagnostic platform.

### SUMMARY OF THE DISCLOSURE

There is thus a need for methods and systems that optimizing a clinician's experience within diagnostic platforms, including the use of artificial intelligence systems that avoid switching from or leaving the diagnostic platform in order to engage with an AI model.

Various embodiments and implementations are directed to artificial intelligence-integrated diagnostic methods and systems. A diagnostic platform receives information about a subject's set of results currently being reviewed by the user via a review window of the diagnostic platform. The system extracts data from the received information to generate contextualized data regarding the set of results, and also receives examination data about the set of results from the user. A trained model of the system analyzes the generated contextualized data and the received examination data to generate diagnostic output about the set of results, and presents the generated diagnostic output to the user via the review window of the diagnostic platform.

According to an aspect, a diagnostic platform method is provided. The method includes: (i) receiving, from one or more data sources, information about a subject's set of results currently being reviewed by a user via a review window of the diagnostic platform; (ii) extracting data from the received information to generate contextualized data regarding the set of results; (iii) selecting an artificial intelligence (AI) module to use based on the generated contextualized data; (iv) packaging and transmitting information to the selected AI module; (v) receiving, via a user interface of the review window of the diagnostic platform, examination data about the set of results from the user; (vi) analyzing, by a trained model, the generated contextualized data and the received examination data to generate diagnostic output about the set of results; and (vii) presenting, via the review window of the diagnostic platform, the generated diagnostic output.

According to an embodiment, the trained model is the selected AI module, and wherein the AI module is selected by an AI Orchestrator of the diagnostic platform.

According to an embodiment, the diagnostic platform is a platform for viewing physiological data for the purposes of the clinician making a diagnosis. According to an embodiment, the diagnostic platform is an ECG platform, echo PACS, or radiology PACS.

According to an embodiment, the received information comprises information of multiple different modalities.

According to an embodiment, the multiple different modalities comprise two or more of text, waveforms, and images.

According to an embodiment, the examination data is text, imaging, video, or audio input.

According to an embodiment, contextual information includes all received information as well as the current state of user interaction with the diagnostic platform.

According to an embodiment, a contextualizer module is responsible for identifying the user experienced based context, available data, and then setting the AI policy module for transmission of data to a subsequent AI module.

According to an embodiment, the contextualizer is specifically trained for the user, and wherein the generated output is tailored to the user's previous interactions, the current usage, and/or available information.

According to an embodiment, the contextualizer can use, entirely or partially, the set of physiological data being reviewed by the user prior results for the same subject from available clinical data, and wherein the generated diagnostic output comprises a summary of the prior results for the subject. According to an embodiment, the returned information includes summaries, descriptions, and measurements and findings about the subject currently being reviewed by the user.

According to an embodiment, the trained model is specifically trained for the user, and wherein the generated diagnostic output is tailored to the user.

According to an embodiment the generated diagnostic output comprises one or more action items, and the method further comprises: receiving feedback from the user via the user interface of the review window, comprising a selection of an action/response; and implementing the selected action/response.

According to an embodiment the generated diagnostic output comprises autocomplete information for the set of results, and the method further comprises: receiving feedback from the user via the user interface of the review window, comprising a selection of the autocomplete information; and displaying the selected autocomplete information as selected.

According to an embodiment, the examination data is received and the generated diagnostic output is presented without the user leaving the review window of the diagnostic platform.

According to an embodiment, the generated output can, for example but not limited to, summary of prior study and lab results, a direct search and retrieval of any parameter associated with prior study and lab results, a summary of similar patients subsequent care pathway, a selection of next action steps such as ordering a test, scheduling follow-ups, referral to a specialist, procedure, medication, discharge, admission, transfer, and so on, or a summary of the current findings in the form of a progress notes.

According to an embodiment, the generated output can be designed to show relevant prior information, next action steps, diagnostic summaries, and information to subsequent care pathways.

According to another aspect is a system for reviewing a subject's data. The system includes: one or more data sources comprising information about a subject; a trained model; a user interface within a review window; and a processor configured to: (i) receive, from the one or more data sources, information about a subject's set of results currently being reviewed by a user via a review window of the system; (ii) extract data from the received information to generate contextualized data regarding the set of results; (iii) selecting an artificial intelligence (AI) module to use based on the generated contextualized data; (iv) packaging and transmitting information to the selected AI module; (v) receive, via the user interface of the review window, examination data about the set of results from the user; (vi) analyze, by the trained model, the generated contextualized data and the received examination data to generate diagnostic output about the set of results; and (vii) direct the user interface to present the generated diagnostic output.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a diagnostic platform method, in accordance with an embodiment.

FIG. 2 is a schematic representation of a diagnostic platform, in accordance with an embodiment.

FIG. 3 is a flowchart of a diagnostic platform method, in accordance with an embodiment.

FIG. 4 is a schematic representation of a diagnostic platform, in accordance with an embodiment.

FIG. 5 is a flowchart of a diagnostic platform method, in accordance with an embodiment.

### DETAILED DESCRIPTION OF EMBODIMENTS

The present disclosure describes various embodiments of artificial intelligence-integrated diagnostic platforms and methods. More generally, Applicant has recognized and appreciated that it would be beneficial to provide a method and system to optimize a clinician's experience with diagnostic platforms. A diagnostic platform receives information about a subject's set of results currently being reviewed by the user via a review window (aka a “study window”) of the diagnostic platform. The system extracts data from the received information to generate contextualized data regarding the set of results, and also receives examination data about the set of results from the user. A trained model of the system analyzes the generated contextualized data and the received examination data to generate diagnostic output about the set of results, and presents the generated diagnostic output to the user via the review window of the diagnostic platform.

The embodiments and implementations disclosed or otherwise envisioned herein can be utilized with any diagnostic platform or other platform utilized by a clinician to review information relevant to a subject or patient. For example, one application of the embodiments and implementations herein is to improve systems such as, e.g., the Philips® IntelliSpace® line of diagnostic and reporting tools (manufactured by Koninklijke Philips, N.V.), among many other products. However, the disclosure is not limited to these devices or systems, and thus the disclosure and embodiments disclosed herein can encompass any artificial intelligence-integrated method, device, or system.

According to an embodiment, the methods and systems described or otherwise envisioned herein utilize an artificial intelligence-integrated diagnostic platform to optimize a clinician's time and efficiency. Currently, AI such as large-language models (LLMs) replace query or pseudo-code user interfaces with a conversation approach, usually in the form of a chat window or an instant message application. The technology enabling this function generally involves a loop, whereby a conversation-like exchange is cumulatively and/or iteratively fed into the LLM, providing additional contexts and triggering further refinement of the response. In some cases, the input can be multi-modal. For example, the user can speak into a microphone, copy/paste text, and attach images, among other modes.

The primary issue with these types of chatbots is that they force a clinician to switch contexts. In contrast, while the methods and systems described or otherwise envisioned herein accept LLM as a commodity, they also provide a method to invoke LLM functionality within a naturalistic use context. Thus, the system automate the context specification, in the background, while also using the actual diagnostic entry window to interact with machine learning (ML) algorithms. Even without a clinician entering any data such as text, the context of the read (for example the automated measurements, relevant patient data such as gender and age group, automated reads, and physiological data) is continually processed and analyzed by the system.

Every statement the clinician contributes then builds on the overall context. This can include the clinician identifying additional results from different studies, thus enabling the clinician to refine the input into the algorithm (which can be a basic ML algorithm, NLP algorithm, decoder architecture, LLM, etc.). Notably, all references to LLMs, AI, and algorithms herein encompass AI/ML approaches that underlie supervised and unsupervised deep learning and linear model development. Each answer (i.e., algorithm output) is continually displayed with specific action points, including but not limited to action points such as accept/reject, “Copy result,” “Append to Progress Note,”, “Append to Patient Record,” and many, many more.

According to an embodiment, the output of the artificial intelligence-integrated diagnostic platform can be continually tuned to each result view and feedback, as how the clinician interacts with the system will provide a training signal for fine-tuning model performance.

Thus, according to an embodiment of the methods and systems described or otherwise envisioned herein, the artificial intelligence-integrated diagnostic platform bypasses explicit invoking of LLM through a chat like interface (for example, a clinician switching windows and typing “Given a diagnosis of right bundle branch block and moderately impaired ejection fraction, what is the recommended treatment?”), and leverages the structured data entry of a diagnostic platform and the mechanism of autocomplete to: (1) invoke an LLM based knowledge search and (2) present a set of best match options via autocomplete suggestions. Through the natural, in-flow usage of a diagnostic platform, the system parses the text as the clinician notes are being entered and provides contextual data given the structured data being presented in the application. Together, the clinician comments and contextual data are formatted into embeddings, which are then used to query the LLM.

The contextualizer component can be trained on the UX of the diagnostic platform, as different components imply different areas of attention. For example, measurements can be made directly on the image or ECG using the mouse-cursor interaction. As the cursor is on the physiological data, the context is that the clinician is “reading” the study; in this case, the results window can show prior results, summaries, and other relevant patient data. If the clinician is typing into the notes and diagnosis window, then she is documenting her diagnosis, and here, a smart “autocomplete” list can be returned. In addition, in the results window, we can see a progress note and subsequent actions populating, as she types. In addition, the clinician notes are not limited to the diagnoses at hand. The clinician is able to type any statement including findings from other studies, lab results, other pertinent information. The role of the orchestrator is to “learn” the context if the clinician is entering a diagnostic statement finding or alternative text. The behavior of AI orchestrator can be refined via the action/accept signals. Finally, the clinician can speed up their analysis by selecting the best statements and uses via accepting the suggestion(s).

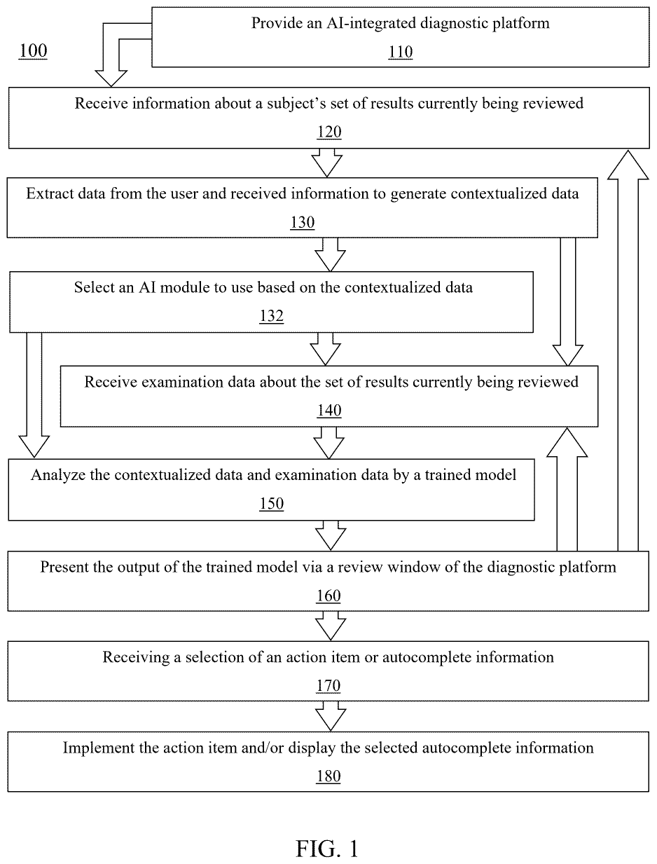

Referring to FIG. 1, in one embodiment, is a flowchart of a method 100 using an artificial intelligence-integrated diagnostic platform. The methods described in connection with the figures are provided as examples only, and shall be understood not to limit the scope of the disclosure. The artificial intelligence-integrated diagnostic platform can be any of the devices or systems described or otherwise envisioned herein. The artificial intelligence-integrated diagnostic platform can be a single device or system, or can be multiple different devices or systems, or can be multiple devices or systems located proximally (e.g., an on-premise system) or distally (e.g., a cloud service provider).

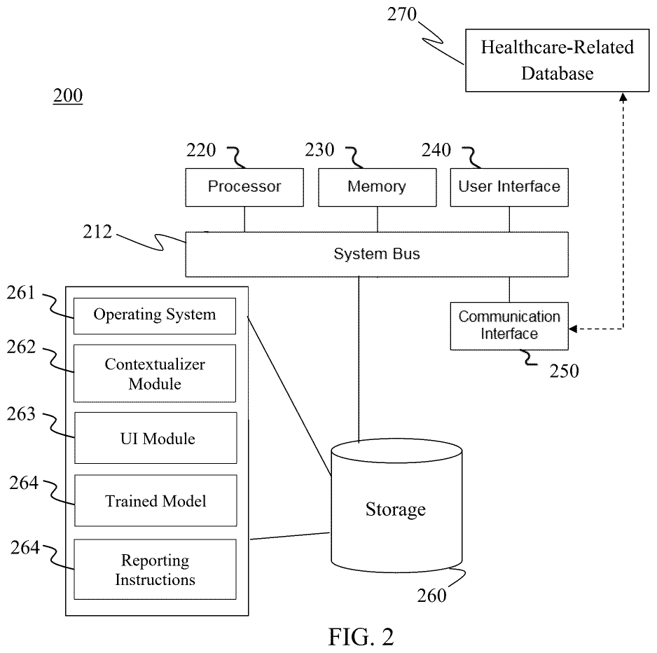

At step 110 of the method, an artificial intelligence-integrated diagnostic platform 200 is provided. Referring to an embodiment of a diagnostic platform 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the diagnostic platform 200 may be different and more complex than illustrated. Additionally, diagnostic platform 200 can be any of the devices described or otherwise envisioned herein. Other elements and components of the diagnostic platform 200 are disclosed and/or envisioned elsewhere herein.

According to an embodiment, the artificial intelligence-integrated diagnostic platform 200 comprises or is in direct or indirect communication with a healthcare-related database 270, such as for example an electronic medical record (EMR) database or system 270, comprising health data or other information for a plurality of patients or subjects. The health data can be any information about the plurality of patients or subjects. According to an embodiment, the information comprises one or more of demographic information about the patient, a diagnosis for the patient, medical history of the patient such as treatment information, lab or test results, and/or any other information. For example, demographic information may comprise information about the patient such as name, age, body mass index (BMI), and any other demographic information. The diagnosis for the patient may be any information about a medical diagnosis for the patient, historical and/or current. The medical history of the patient may be any historical admittance or discharge information, historical treatment information, historical diagnosis information, historical exam or imaging information, and/or any other information (although in some embodiments, a patient's medical history may not be available). The lab or test results may be, for example, an analysis of blood gases, electrolytes, biomarkers, ECG results, echocardiogram results, ultrasound results, CT results, MRI results, and/or any other types of lab tests or testing. Many other forms and types of patient health data are possible.

At step 120 of the method, a clinician is reviewing a subject's current study in a review window of the artificial intelligence-integrated diagnostic platform 200. The clinician can be any user such as a physician, nurse, healthcare professional, or any other user. The subject can be a patient or any other person for which results or other data or information is available for review in the platform. The artificial intelligence-integrated diagnostic platform 200 can be any of the platforms, devices, or systems described or otherwise envisioned herein. At step 120 of the method, the artificial intelligence-integrated diagnostic platform 200 receives information about the subject's set of results currently being reviewed by a user via a review window the diagnostic platform. The information can be received or otherwise obtained from one or more data sources, such as the healthcare-related database or system 270, among many other sources.

The information about a subject's set of results may be, for example, anything relevant to the subject and/or the set of results. For example, the information may be historical information about the subject, such as previous results or testing, or historical medical treatment. The information may be demographic information about the subject. The information may be more broadly related to the subject's set of results, such as results from similar subjects, and so on. Many other types of information are possible.

At step 130 of the method, the artificial intelligence-integrated diagnostic platform 200 extracts data from the received information in order to generate contextualized data regarding the set of results. The data may be extracted using a wide variety of methods. Once extracted, the information may be utilized immediately and/or may be stored in local and/or remote memory.

According to an embodiment, the artificial intelligence-integrated diagnostic platform 200 comprises a contextualizer module configured to extracts data from the received information. The contextualizer module may, for example, be implemented as a module or component of the processor 220 of the system, such as by software or an algorithm. However, the system is not limited to the implementation of the contextualizer module, and thus other processes for extracting data are possible.

According to an embodiment, the contextualizer comprises or utilizes a set of data interfaces to connect to additional data sources from which information about the subject and/or the results can be obtained. As described herein, the additional data sources can be anything that can provide or comprise information about the subject and/or results being reviewed. The contextualizer module also extracts data from the currently viewed data in the review window of the diagnostic platform. The data can comprise one or more modalities, such as text, waveforms, images, and more. According to an embodiment, contextual information includes some or all of the received information as well as the current state of user interaction with the diagnostic platform. The extracted data is transformed into embeddings for subsequent processing.

According to an embodiment, the contextualizer begins functioning as soon as results are accessed by the clinician. Even while the results are being reviewed, these and additional data are encoded by the contextualizer and fed into the AI model as described or otherwise envisioned herein.

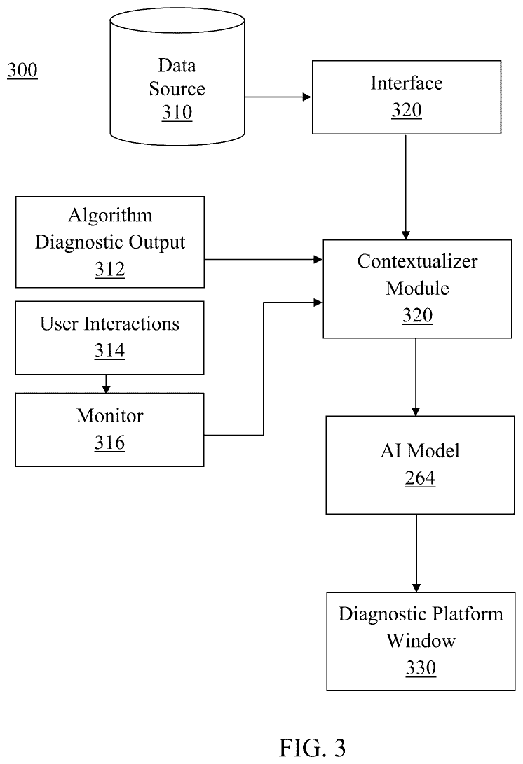

Referring to FIG. 3, in one embodiment, is a diagram of data and processing flow with regard to the artificial intelligence-integrated diagnostic platform 200 and methods thereof. The system comprises, for example, one or more data sources 310 and one or more interfaces 320 that interface between the data sources and the contextualizer module 320. The contextualizer module can also receive as input: data from a diagnostic platform algorithm (Algorithm Diagnostic Output 312), as well as input by the user by user experience related controls (User interactions 314).

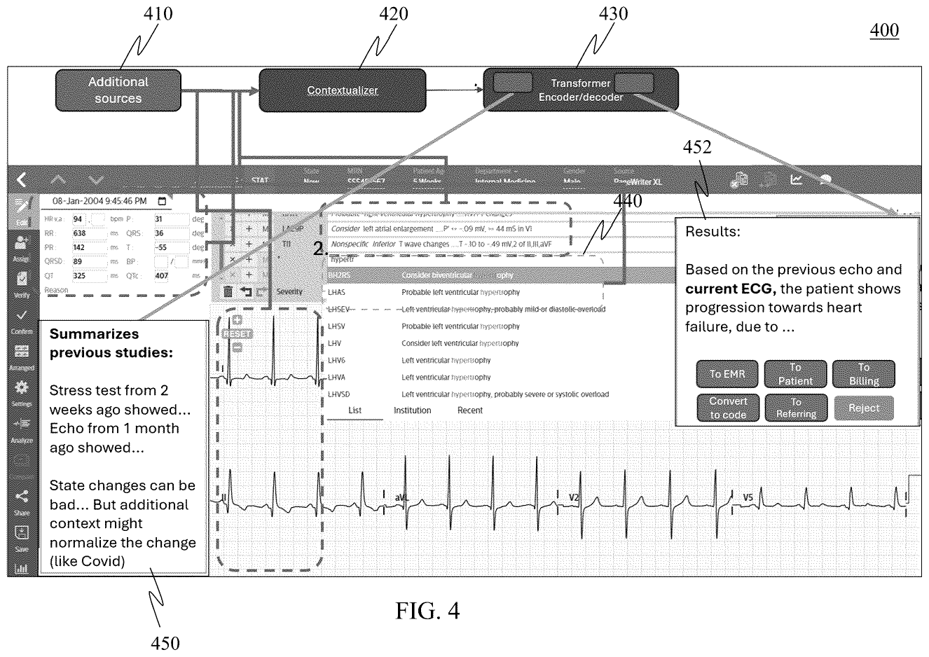

Referring to FIG. 4, in one embodiment, is a diagram of data sources for the artificial intelligence-integrated diagnostic platform 200 and methods thereof. The system comprises, for example, one or more data sources 410, which can be any of the data sources described or otherwise envisioned herein. For example, the system pulls, obtains, or otherwise receives data from sources such as the parameters of the testing, historical information about the patient, the waveforms (in this ECG example), and more. Many other data sources are possible. Notably, the platform may be an ECG platform, an echo PACS, a radiology PACS, or any other suitable platform.

At step 132 of the method, the artificial intelligence-integrated diagnostic platform 200 selects an artificial intelligence (AI) module to use for downstream analysis (as described or otherwise envisioned herein) based at least in part on the generated contextualized data. Thus, the platform can comprise or be in communication with a plurality of different possible AI modules. These AI modules can be any AI platform as described or otherwise envisioned herein. The platform can select or identify an AI module to utilize, based at least in part on the generated contextualized data, in a wide variety of different ways.

According to an embodiment, the artificial intelligence-integrated diagnostic platform 200 comprises an AI Orchestrator that selects an AI module to use for downstream analysis (as described or otherwise envisioned herein) based at least in part on the generated contextualized data. The AI Orchestrator can be an AI policy module for identifying context and configuring the data to transmit to a subsequent AI module. According to an embodiment, the AI Orchestrator is specifically trained for the user, and wherein the generated output is tailored to the user's previous interactions, the current usage, and available information. According to an embodiment, the AI Orchestrator can use, entirely or partially, the set of physiological data being reviewed by the user and prior results for the same subject from available clinical data.

The AI orchestrator can be trained on the UX of the diagnostic platform, as different components imply different areas of attention. For example, measurements can be made directly on the image or ECG using the mouse-cursor interaction. As the cursor is on the physiological data, the context is that the clinician is “reading” the study; in this case, the results window can show prior results, summaries, and other relevant patient data. If the clinician is typing into the notes and diagnosis window, then she is documenting her diagnosis, and here, a smart “autocomplete” list can be returned. In addition, in the results window, we can see a progress note and subsequent actions populating, as she types.

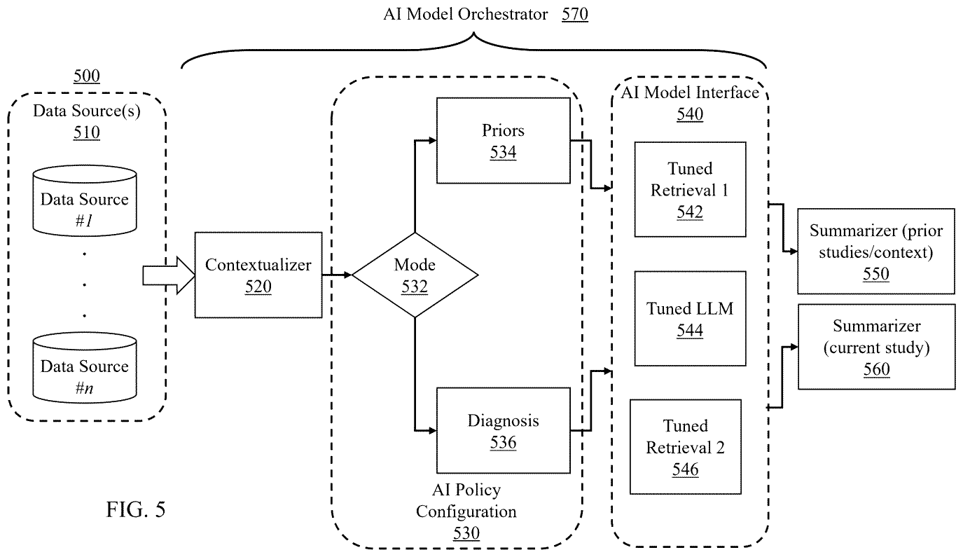

Referring to FIG. 5, in one embodiment, is a schematic representation 500 of an AI model usage policy and execution flow. The system can include, for example, a configuration tool to establish AI model policy. The system can offer modes for strict information retrieval (embeddings used for query/key match) to return actual data, all the way to enabling generative AI (i.e. decoder/LLM technology) to enable summary creation. As shown in the embodiment of FIG. 5, the schematic representation 500 of the system also shows data sources 510 (including data source #1, . . . to data source #n), the contextualizer module 520, to the AI configuration policy 520 with a mode 532 and prior data 534 and current data/diagnosis 536. The system also comprises an AI model interface API/access/storage 540 with, for example, some tuned models 542, 544, and 546. There can be more tuned models, provided by 1^{st} and 3^{rd} parties. The modules 520, 530, and 540 comprise the AI Orchestrator module. The output can include a summarizer (and output panel) of prior studies and data or context 550, and a summarizer (and output panel) of the current data or study 560.

According to an embodiment, once the AI module to be used is selected or identified, the system packages and transmits information, such as the contextualized data among other possible data, to the selected or identified AI module. The information can be packaged using any method for packaging data for transmission. The packaged data can be transmitted using any method for transmitting data via a wired and/or wireless network.

At step 140 of the method, the artificial intelligence-integrated diagnostic platform 200 receives examination data about the set of results currently being reviewed in the platform. The examination data is received from the clinician via a user interface of the review window of the diagnostic platform. Notably, the user interface can receive examination data in numerous ways, including but not limited to text entry, pull-down selection, audio, and more.

According to an embodiment, the artificial intelligence-integrated diagnostic platform 200 comprises a user interface (UI) module configured to allow the entry of the examination data about the set of results currently being reviewed in the platform. The UI module may, for example, be implemented as a module or component of the processor 220 of the system, such as by software or an algorithm. However, the system is not limited to the implementation of the UI module, and thus other processes for data entry by the user are possible.

According to an embodiment, the UI module builds on the existing diagnostic platform. User-entered statements are monitored and processed in real time by the contextualizer module (and by the AI module as described herein). The examination data may be diagnostic code (i.e., structured) or freeform (i.e., unstructured data) text entry. According to an embodiment, the UI control point is at the diagnostic editor. For example, whatever the clinician types is subsequently encoded and contextualized with available information. Thus, the clinician can type in a diagnosis, or even a counter-factual prompt. These embeddings are continuously sent to the AI model (as described herein), with the results displayed in the output panels (as described herein). In other words, the UI module and the available information (which can include data outside of the ECG system) are continually processed by the AI model. From the user's point of view, the clinician engages with the AI/LLM seamlessly, where they would normally enter text.

According to an embodiment, the examination data entered into the system is immediately provided to the contextualizer module for analysis as described or otherwise envisioned herein.

Referring again to FIG. 3 is a diagram of data and processing flow with regard to the artificial intelligence-integrated diagnostic platform 200 and methods thereof. The system comprises, for example, a UI module 314 (“user interactions 314”) that allows for entry of examination data, which may be text or any other modality. According to an embodiment, the system also comprises a monitor which can cause the system to pause (such as the functionality of the contextualizer and/or trained model) when the user is entering data.

Referring again to FIG. 4, in one embodiment, is a diagram of data sources for the artificial intelligence-integrated diagnostic platform 200 and methods thereof. The system comprises, for example, a UI module 440 that allows the clinician to enter examination data. Many other formats of the UI module and thus the user interface are possible.

The results of data extract and embedding by the contextualizer module are then provided to the trained model 264 of the artificial intelligence-integrated diagnostic platform 200. The results can be provided to the trained model as they are generated, or they can be provided in batches. Many methods for providing the results are possible.

At step 150 of the method, the trained model 264 of the artificial intelligence-integrated diagnostic platform 200 analyzes the generated contextualized data and the received examination data (and/or the results of that data after analysis by the contextualizer) to generate diagnostic output about the set of results. The trained model 264 can be any model that can be trained to utilize the input to generate the output, as described or otherwise envisioned herein. For example, the model can be a neural network or other trained machine learning model, for example a convolutional neural network (CNN) or a transformer network or other neural network. Thus, according to an embodiment, the artificial intelligence-integrated diagnostic platform 200 comprises a trained model 264 that receives the input data (e.g., the generated contextualized data and the received examination) and outputs the various output as described or otherwise envisioned herein.

According to an embodiment, the trained model comprises a transformer architecture with an encoder/decoder. There can be, for example, an umbrella deep learning network comprising multiple classes of summarizers, object recognition, and so on. The trained model or AI system also comprises one or more large language model architectures, although according to an embodiment the trained model or AI system may be in communication with a local or remote LLM architecture or service which is separate from the trained model.

According to an embodiment, the trained model may comprise one or more sub-modules. The system thus comprises a model that is tuned to act as a query interface, identifying relevant information from the same patient's previous labs and studies. According to an embodiment, another submodule can generate a summary for output. Since there can be multiple groups of audiences (e.g., other experts, the primary care physician, the patient, etc.), output can be tailored accordingly. Thus, a single or multiple models can be leveraged, with performance of each optimized separately.

According to an embodiment, the generated output can, for example but not limited to, summary of prior study and lab results, a direct search and retrieval of any parameter associated with prior study and lab results, a summary of similar patients subsequent care pathway, a selection of next action steps such as ordering a test, scheduling follow-ups, referral to a specialist, procedure, medication, discharge, admission, transfer, and so on, or a summary of the current findings in the form of a progress notes. According to an embodiment, the generated output can be designed to show relevant prior information, next action steps, diagnostic summaries, and information to subsequent care pathways. Other output is possible.

Referring to FIG. 3, is a diagram of data and processing flow with regard to the artificial intelligence-integrated diagnostic platform 200 and methods thereof. The system comprises, for example, an AI model 264, which can be any of the models or embodiments described or otherwise envisioned herein, and can comprise one or more sub-modules or components that perform one or more respective functions of the system.

Referring again to FIG. 4, in one embodiment, is a diagram of data sources for the artificial intelligence-integrated diagnostic platform 200 and methods thereof. The system comprises, for example, an AI model 430 (transformer architecture with encoder/decoder).

The output of the trained model 264 of the artificial intelligence-integrated diagnostic platform 200 can then be provided to the user. Thus, according to an embodiment, the output of the trained model is provided to the user via a user interface of the system, such as a review window or interface of the diagnostic platform. The output can be provided to the clinician as they are generated, or they can be provided in batches. Many methods for providing the output to the user interface are possible.

Thus, at step 160 of method 100 in FIG. 1, the generated diagnostic output from the trained model is presented to the clinician via a review window or interface 240 of the artificial intelligence-integrated diagnostic platform 200. The generated diagnostic output can be any of the information as described or otherwise envisioned herein. The system may provide the information to a user via any mechanism, including but not limited to the visual display. The information may be communicated by wired and/or wireless communication to another device. For example, the system may communicate the information to a monitor, screen, mobile phone, computer, laptop, wearable device, and/or any other device configured to allow display and/or other communication of the information in the diagnostic platform.

According to an embodiment, there can be multiple output panels on the user interface to display the generated diagnostic output. For example, according to one embodiment, there can be at least two panels, a first panel summarizing data from prior labs and studies, and a second one integrating and summarizing results that include the current study. An output panel can comprise multiple options for documenting and correcting the results. It can include tailoring the summary text for audience, or parsing into structured codes, or further streamlined for billing, or rejected. The clinician can edit and is in full control of how to handle the information.

Referring to FIG. 3, is a diagram of data and processing flow with regard to the artificial intelligence-integrated diagnostic platform 200 and methods thereof. The system comprises, for example, a user interface 330 which can be, for example, a diagnostic platform window.

Referring again to FIG. 4, in one embodiment, is a diagram of data sources for the artificial intelligence-integrated diagnostic platform 200 and methods thereof. The system comprises, for example, a first output panel 450 that summarizes data (by the trained AI model) from prior labs and studies, and a second output panel 452 that summarizes data and results from the current study. Other embodiments are possible.

At step 170 of the method, the clinician interacts in some way with the generated diagnostic output of the trained AI model, which was provided via the user interface of the artificial intelligence-integrated diagnostic platform 200. For example, the diagnostic output may comprise one or more autocomplete suggestions, which the clinician may select or otherwise identify. As another example, the diagnostic output may comprise an action item or other response that the clinician can select using any method for selection via a user interface.

Referring to FIG. 4, the second output panel 452 summarizes data and results from the current study, and the clinician can interact with the information presented in the panel. As an example, the clinician can select any of the presented information, including one of the action items. As another example, the clinician can edit any of the presented information. Other embodiments are possible.

At step 180 of the method, the artificial intelligence-integrated diagnostic platform 200 implements the selected action item and/or displays the selected autocomplete information. This can include, for example, saving the item or information to memory, or causing the diagnostic platform to take an action. Many other embodiments are possible.

According to an embodiment, the artificial intelligence-integrated diagnostic platform 200 leverages data internal to the diagnostic product (e.g., measurements, algorithmic findings, and waveforms in the current ECG). These data are collected and converted into embeddings, which provides the inputs needed to power the AI-based applications. These applications can include next-diagnostic code retrieval, the ability to create anti-code sets to avoid suggesting diametrically opposite codes, to engage in information retrieval, and to leverage decoder architectures to generate summaries, among other diagnostic outputs.

According to an embodiment of an ECG platform, for example, the input information can include the current ECG and all prior ECGs. The embeddings can be processed to generate clinical trends, more precise information retrieval and more accurate diagnostic suggestions. The system can create cross-diagnostic embeddings to submit to a decoder architecture (i.e. large language model), to generate proper context and prompts to elicit generative summaries. The embeddings can include the current ECG, current and prior ECGs, prior studies and results (for example but not limited to intra-procedure information and echocardiograms), in various combinations. In this way, available information is submitted to a pre-trained LLM to generate summaries. In another embodiment, the architecture can be implemented separately for summarizing prior results, progress notes (i.e. summary of current diagnoses), or both, among other output.

According to an embodiment, a speech-to-text module can be integrated at the point of the diagnostic statement editor. The clinician can speak the text or diagnostic statements, which can then be embedded and sent to the AI model for inference.

Referring again to FIG. 5, in one embodiment, is a schematic representation 500 of an AI model usage policy and execution flow. The system can include, for example, a configuration tool to establish AI model policy. The system can offer modes for strict information retrieval (embeddings used for query/key match) to return actual data, all the way to enabling generative AI (i.e. decoder/LLM technology) to enable summary creation. As shown in the embodiment of FIG. 5, the schematic representation 500 of the system also shows data sources 510 (including data source #1, . . . to data source #n), the contextualizer module 520, to the AI configuration policy 520 with a mode 532 and prior data 534 and current data/diagnosis 536. The system also comprises an AI model interface API/access/storage 540 with, for example, some tuned models 542, 544, and 546. There can be more tuned models, provided by 1^{st} and 3^{rd} parties. The modules 520, 530, and 540 comprise the AI Orchestrator module. The output can include a summarizer (and output panel) of prior studies and data or context 550, and a summarizer (and output panel) of the current data or study 560.

According to an embodiment, the system can swap in/swap out AI models, or add models, to expand on additional use cases (e.g., outside of summarizers or data retrieval). For example, the system might comprise a diffusion model, which can generate an exemplar image of the physiological finding for direct comparison between the actual and ideal case. In much the same way as shown in FIG. 5, the system can comprise configuration such that it switches between a diffusion model (generative images) to retrieval of actual non-textual data using semantic search (retrieval, not generative summarization).

According to an embodiment, the system can comprise a key-press shortcut to specifically engage the AI model use, such as an LLM-based search with summarization or a vector store approach to return an exact result, or search for specific measurements only. This shortcut can be a part of the diagnostic statement editor, thus giving the clinician more control and flexibility while not leveraging another application or workflow. Essentially, insights are pulled and presented to the clinician, without disrupting their workflow or having them take additional steps.

According to an embodiment, therefore, the system comprises an engine that can be adapted to the specific diagnostic platform. A given patient's structured and unstructured data—under current view—can be embedded and packaged for consumption by a host of deep learning technologies, specifically the state-of-the-art transformer (encoder/decoder) and large-language model (transformer decoder) architectures. Access to data sources can be achieved via specific data interfaces, or ETL designed for extensive interoperability within diagnostic review products. These data, relevant to the patient, can also be included in the embedding, providing additional context with which to refine the results returned by the transformer/LLM architectures. Further, depending on the type of data being embedded (prior+current versus prior), the system can select the appropriate display, for influencing the current diagnosis or to summarize results into a progress note.

Referring again to FIG. 2 is a schematic representation of an artificial intelligence-integrated diagnostic platform 200. System 200 may be any of the devices or systems described or otherwise envisioned herein, and may comprise any of the components described or otherwise envisioned herein. It will be understood that FIG. 2 constitutes, in some respects, an abstraction and that the actual organization of the components of system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may also comprise or be in direct or indirect communication with an electronic medical record system and/or an electronic medical records (EMR) database from which the information about patients may be obtained or received. For example, EMR database may comprise information about previous results, diagnostic information, or any other information about a patient. According to an embodiment, the electronic medical record system may be a local or remote database and is in direct and/or indirect communication with system 200. Thus, according to an embodiment, the system comprises an electronic medical record database or system.

According to an embodiment, storage 260 of system 200 may store one or more algorithms, modules, and/or instructions to carry out one or more functions or steps of the methods described or otherwise envisioned herein. For example, storage 260 may comprise, among other instructions or data, a contextualizer module 262, UI module 263, a trained AI model 264, and/or reporting instructions 265.

According to an embodiment, contextualizer module 262 directs the system to extract data from the information received from the one or more data sources and/or input from the clinician about the current study being reviewed. The contextualizer module may, for example, be implemented as a module or component of the processor 220 of the system, such as by software or an algorithm. However, the system is not limited to the implementation of the contextualizer module, and thus other processes for extracting data are possible. According to an embodiment, the contextualizer comprises or utilizes a set of data interfaces to connect to additional data sources from which information about the subject and/or the results can be obtained. As described herein, the additional data sources can be anything that can provide or comprise information about the subject and/or results being reviewed. The contextualizer module also extracts data from the currently viewed data in the review window of the diagnostic platform. The data can comprise one or more modalities, such as text, waveforms, images, and more. The extracted data is transformed into embeddings for subsequent processing. According to an embodiment, the contextualizer begins functioning as soon as results are accessed by the clinician. Even while the results are being reviewed, these and additional data are encoded by the contextualizer and fed into the AI model as described or otherwise envisioned herein.

According to an embodiment, UI module 263 directs the system to allow the entry of examination data about the set of results currently being reviewed in the platform. The UI module may, for example, be implemented as a module or component of the processor 220 of the system, such as by software or an algorithm. However, the system is not limited to the implementation of the UI module, and thus other processes for data entry by the user are possible. As described herein, the platform 200 receives examination data about the set of results currently being reviewed in the platform. The examination data is received from the clinician via a user interface of the review window of the diagnostic platform. Notably, the user interface can receive examination data in numerous ways, including but not limited to text entry, pull-down selection, audio, and more. According to an embodiment, the UI module builds on the existing diagnostic platform. User-entered statements are monitored and processed in real time by the contextualizer module (and by the AI module as described herein). The examination data may be diagnostic code (i.e., structured) or freeform (i.e., unstructured data) text entry. According to an embodiment, the UI control point is at the diagnostic editor. For example, whatever the clinician types is subsequently encoded and contextualized with available information. Thus, the clinician can type in a diagnosis, or even a counter-factual prompt. These embeddings are continuously sent to the AI model (as described herein), with the results displayed in the output panels (as described herein). In other words, the UI module and the available information are continually processed by the AI model. From the user's point of view, the clinician engages with the AI/LLM seamlessly, where they would normally enter text.

According to an embodiment, the trained AI model 264 of the system is trained to analyze the received input, including but not limited to the generated contextualized data and the received examination data (and/or the results of that data after analysis by the contextualizer) to generate diagnostic output about the set of results. The trained model 264 can be any model that can be trained to utilize the input to generate the output, as described or otherwise envisioned herein. For example, the model can be a neural network or other trained machine learning model, for example a convolutional neural network (CNN) or a transformer network or other neural network. Thus, according to an embodiment, the system 200 comprises a trained model that receives the input data and outputs the diagnostic output, as described or otherwise envisioned herein.

According to an embodiment, reporting instructions 265 directs the system to provide the output of the system—such as the diagnostic output of the AI model—to a patient, clinician, or to another device or system. The provided output can be any of the information as described or otherwise envisioned herein. The system may provide the information to a user via any mechanism, including but not limited to a visual display. The information may be communicated by wired and/or wireless communication to another device. For example, the system may communicate the information to a monitor, screen, mobile phone, computer, laptop, wearable device, and/or any other device configured to allow display and/or other communication of the information. According to an embodiment, the system displays some or all of the output in the diagnostic platform, such as in a review window of the platform.

According to an embodiment, system 200 is configured to process many thousands or millions of datapoints in the input data used to train the AI model 264. For example, generating a functional and skilled trained neural network encoder from a corpus of training data requires processing of millions of datapoints from input data and generated features. This can require millions or billions of calculations to generate a novel trained neural network encoder from those millions of datapoints and millions or billions of calculations. As a result, each trained neural network encoder is novel and distinct based on the input data and parameters of the model, and thus improves the functioning of the system. Generating a functional and skilled trained neural network encoder comprises a process with a volume of calculation and analysis that a human brain cannot accomplish in a lifetime, or multiple lifetimes.

Further, the methods and systems described or otherwise envisioned herein are specific methods or systems that improve diagnostic platform technology, rather than abstractly covering results without regard to a specific process or machinery for achieving those results. Here, the method and system for generating the diagnostic output by the trained AI model is a specific method and system that improves diagnostic platform technology. Specifically, the method and system comprises an improved diagnostic platform that generates and provides the diagnostic output in the review window of the diagnostic platform, thus avoiding the requirement in prior art systems that the clinician/reviewer move to another window to perform the interaction with the trained AI model. In other words, the user interview (e.g., the review window of the diagnostic platform) is an improved user interface that provides information in a significantly more time-efficient and computationally efficient manner. This saves the clinician time and energy, and avoids distraction, thus also improving healthcare by the clinician. Prior art diagnostic platforms do not implement a trained AI model—such as a generative or LLM model—within the review window of the platform, instead requiring the clinician leave the platform to engage with the trained AI model. Thus, the results are generated using multiple different user interfaces, thereby requiring both increased clinician time/effort and computational time/effort. In contrast, the methods and systems described or otherwise envisioned herein implement the trained AI model—such as a generative or LLM model—within the review window of the platform, which is more time- and computationally efficient. Thus, the processes described or otherwise envisioned herein improve the functionality of the diagnostic platform as it requires fewer user interfaces and less time or computational effort generate the diagnostic output. The methods and systems thus require specific, technological means—i.e., the contextualizer, the trained AI model, the UI module—that in turn provides a technological improvement to the diagnostic platform. Thus, the methods and systems recite a technological solution to a technological problem.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A diagnostic platform method (100), comprising:
receiving (120), from one or more data sources, information about a subject's set of results currently being reviewed by a user via a review window of the diagnostic platform;
extracting (130) data from the received information to generate contextualized data regarding the set of results;
receiving (140), via a user interface of the review window of the diagnostic platform, examination data about the set of results from the user;
analyzing (150), by a trained model (264), the generated contextualized data and the received examination data to generate diagnostic output about the set of results; and
presenting (160), via the review window of the diagnostic platform, the generated diagnostic output.

2. The method of claim 1, further comprising the step of selecting (132) an artificial intelligence (AI) module to use for analysis based on the generated contextualized data, wherein the trained model is the selected AI module.

3. The method of claim 1, wherein the diagnostic platform is a platform for viewing physiological data for a user to make a diagnosis.

4. The method of claim 1, wherein the received information comprises information of multiple different modalities, the multiple different modalities comprising two or more of text, waveforms, and images.

5. The method of claim 1, wherein the examination data is text, imaging, video, or audio input.

6. The method of claim 1, wherein the received information about the set of results being reviewed by the user comprises prior results for the subject, and wherein the generated diagnostic output comprises a summary of the prior results for the subject.

7. The method of claim 6, wherein the summary further comprises information about the subject's set of results currently being reviewed by the user.

8. The method of claim 1, wherein the trained model is specifically trained for the user, and wherein the generated diagnostic output is tailored to the user.

9. The method of claim 1, wherein the generated diagnostic output comprises one or more actions or responses for selection, and wherein the method further comprises:
receiving (170), from the user via the user interface of the review window, a selection of an action or response; and
implementing (180) the selected action or response.

10. The method of claim 9, wherein the response comprises autocomplete information for the set of results.

11. The method of claim 1, wherein the examination data is received and the generated diagnostic output is presented without the user leaving the review window of the diagnostic platform.

12. A system (200) for reviewing a subject's data, comprising:
one or more data sources (270) comprising information about a subject;
a trained model (264);
a user interface (240) within a review window; and
a processor (200) configured to: (i) receive, from the one or more data sources, information about a subject's set of results currently being reviewed by a user via a review window of the system; (ii) extract data from the received information to generate contextualized data regarding the set of results; (iii) receive, via the user interface of the review window, examination data about the set of results from the user; (iv) analyze, by the trained model, the generated contextualized data and the received examination data to generate diagnostic output about the set of results; and (iv) direct the user interface to present the generated diagnostic output.

13. The system of claim 12, wherein the processor is further configured to select an artificial intelligence (AI) module to use for analysis based on the generated contextualized data, and wherein the trained model is the selected AI module.

14. The system of claim 12, wherein the received information about the set of results being reviewed by the user comprises prior results for the subject, and wherein the generated diagnostic output comprises a summary of the prior results for the subject.

15. The system of claim 12 wherein the examination data is received and the generated diagnostic output is presented without the user leaving the user interface within the review window.
